(19) United States
(12) Patent Application Publication
(10) Pub. No.: US 2025/0000926 A1
(43) Pub. Date: Jan. 2, 2025

(54) METHOD FOR PREVENTING, IMPROVING OR TREATING INFLAMMATORY SKIN DISEASE COMPRISING A COMPOSITION COMPRISING A FERMENTED ANGELICA GIGAS EXTRACT

(71) Applicant: Sooy-K Co., Ltd., Seongnam-si (KR)

(72) Inventors: Youn Ok JUNG, Namwon-si (KR); Ji Sun CHUNG, Seongnam-si (KR); Kyoung Mo CHUNG, Seoul (KR); Seo A JUNG, Seongnam-si (KR); Ga Hyeon SONG, Seongnam-si (KR)

(21) Appl. No.: 18/451,685

(22) Filed: Aug. 17, 2023

(30) Foreign Application Priority Data
Jun. 30, 2023 (KR) 10-2023-0084735
Jul. 17, 2023 (KR) 10-2023-0092295

(51) Int. Cl. A61K 36/232; A61P 29/00; A61P 31/00

(52) U.S. Cl. CPC A61K 36/232; A61P 29/00; A61P 31/00; A61K 2236/15; A61K 2236/19; A61K 2236/333; A61K 2236/35

(57) ABSTRACT

A method for preventing, improving or treating inflammatory skin diseases includes the step of administering a composition comprising a fermented Angelica gigas extract as an active ingredient. The fermented Angelica gigas extract has a significantly enhanced content of the main components of Angelica gigas, resulting in significantly increased antimicrobial and anti-inflammatory activities, so it can be effectively used for preventing, improving or treating inflammatory skin diseases.

### CROSS-REFERENCE TO RELATED APPLICATION

This application is based on and claims priority from Korean Patent Application No. 10-2023-0084735 filed on Jun. 30, 2023; and Korean Patent Application No. 10-2023-0092295 filed on Jul. 17, 2003 in the Korean Intellectual Property Office, the content of which is incorporated herein by reference in there entireties.

### BACKGROUND OF THE INVENTION

### Field of the Invention

The present invention relates to a method for preventing, improving or treating inflammatory skin diseases that contains the step of administering a composition comprising a fermented Angelica gigas extract as an active ingredient, and a method for preparing the same.

### Background of the Related Art

Most diseases, including skin disorders, are accompanied by an inflammatory response. The inflammatory response refers to the mechanism by which the body or tissue tries to restore and regenerate the damaged area when subjected to physical actions, chemical substances, bacterial infections, or any other pathological changes. Once a stimulus is applied, local blood vessel activators are released, leading to increased vascular permeability and inducing inflammation. The initial inflammation that occurs in response to injury is a normal reaction aimed at facilitating the recovery of the damaged site. However, prolonged and continuous inflammatory responses over an extended period can make the recovery of the affected area difficult, cause harm to surrounding cells, and lead to continuous damage to neighboring tissues, possibly resulting in the development of diseases such as cancer. Therefore, it is known that tissues displaying inflammatory responses should be treated with appropriate anti-inflammatory agents to promote rapid recovery.

Representative inflammatory skin diseases include atopic dermatitis, acne, contact dermatitis, psoriasis, and other conditions mediated by immune cells. Steroidal anti-inflammatory agents are commonly used to improve these inflammatory skin diseases, but they often exhibit significant side effects, leading to limitations in their use. Therefore, in the cosmetics industry, there is a growing trend of researching non-steroidal anti-inflammatory agents or natural antimicrobial agents derived from natural substances to improve these skin conditions.

Korean Angelica gigas Nakai has been used as a traditional medicinal herb for a long time. Its main pharmacological components are known to be decursin and decursinol. Angelica gigas promotes blood circulation, provides nutrients to the skin, exhibits anti-inflammatory and antimicrobial effects, acts as an anti-allergic agent, and contains various organic substances such as vitamins. It is thus known to be effective in inhibiting melanin formation, making it beneficial for pigmented skin conditions like freckles and melasma. Additionally, it is believed to help soothe the skin, enhance elasticity, promote blood circulation, and contribute to anti-aging effects, resulting in healthier and more radiant skin.

The inventors have confirmed that the fermented Angelica gigas extract contains a higher concentration of major components such as decursin, decursinol angelate, decursinol, or nodakenin compared to general solvent extracts and exhibits excellent antimicrobial and anti-inflammatory effects for improving itching, thereby completing the present invention.

### SUMMARY OF THE INVENTION

Accordingly, it is an object of the present invention to provide a method for preventing, improving or treating inflammatory skin diseases that comprises the step of administering a composition comprising a fermented Angelica gigas extract as an active ingredient.

It is still further another object of the present invention to provide an antimicrobial composition including a fermented Angelica gigas extract as an active ingredient.

It is still further another object of the present invention to provide an anti-inflammatory composition including a fermented Angelica gigas extract as an active ingredient.

It is still further another object of the present invention to provide a method for preparing a fermented Angelica gigas extract with an enhanced content of main components that comprises the steps of: (a) grinding Angelica gigas; (b) fermenting the ground Angelica gigas by inoculating with a fermentation strain; and (c) extracting the extract by adding a solvent after drying the fermented product obtained in the above step (b).

To achieve the above objects, the present invention provides a method for preventing, improving or treating an inflammatory skin disease, the method comprising administering a composition comprising a fermented Angelica gigas extract as an active ingredient to a subject in need thereof.

The present invention also provides an antimicrobial composition including a fermented Angelica gigas extract as an active ingredient.

The invention also provides an anti-inflammatory composition containing fermented Angelica gigas extract as an active ingredient.

The present invention also provides a method for preparing a fermented Angelica gigas extract with enhanced content of main components that comprises the steps of: (a) grinding Angelica gigas; (b) fermenting the ground Angelica gigas by inoculating with a fermentation strain; and (c) extracting the extract by adding a solvent after drying the fermented product obtained in the above step (b).

The composition comprising a fermented Angelica gigas extract according to the present invention has a significantly enhanced content of the main components of Angelica gigas, resulting in significantly increased antimicrobial and anti-inflammatory activities, so it can be effectively used for preventing, improving or treating inflammatory skin diseases.

### BRIEF DESCRIPTION OF THE DRAWINGS

FIG. 1 shows a comparison of the forms of Angelica gigas according to the degree of grinding.

FIG. 2 presents the process of preparing the extract according to the degree of grinding and the concentration of the solvent.

FIG. 3 shows a comparison of concentrated powder extracts according to the degree of grinding and the concentration of the solvent.

FIG. 4 presents the microscopic images of fermentation strains according to the fermentation method.

FIG. 5 presents a comparison of the forms of Angelica gigas after fermentation with different strains.

FIG. 6 presents a comparison of the concentrated powder extracts of Angelica gigas fermented with different strains.

FIGS. 7A-7H provides the results of HPLC analysis on the fermented Angelica gigas extracts according to the fermentation method and the fermentation strain: standard curves of the four different components of Angelica gigas (A); an ethanolic Angelica gigas extract (B); a fermented Angelica gigas extract obtained by solid-state fermentation with Bacillus subtilis under alternating temperature conditions for 9 days (C); a fermented Angelica gigas extract obtained by solid-state fermentation with Lactobacillus casei at 30° C. for 9 days (D); a fermented Angelica gigas extract obtained by solid-state fermentation with Saccharomyces cerevisiae under alternating temperature conditions for 9 days (E); a fermented Angelica gigas extract obtained by liquid-state fermentation with Bacillus subtilis for 1 day (F); a fermented Angelica gigas extract obtained by liquid-state fermentation with Lactobacillus casei at 30° C. for 1 day (G); and an Angelica gigas extract fermented obtained by liquid-state fermentation with Saccharomyces cerevisiae under alternating temperature conditions for 2 days (H).

FIG. 8 shows the measurement results of cell viability after treating with the fermented Angelica gigas extracts at different concentrations.

FIG. 9 presents the measurements results of NO (nitric oxide) production after treating with the fermented Angelica gigas extract.

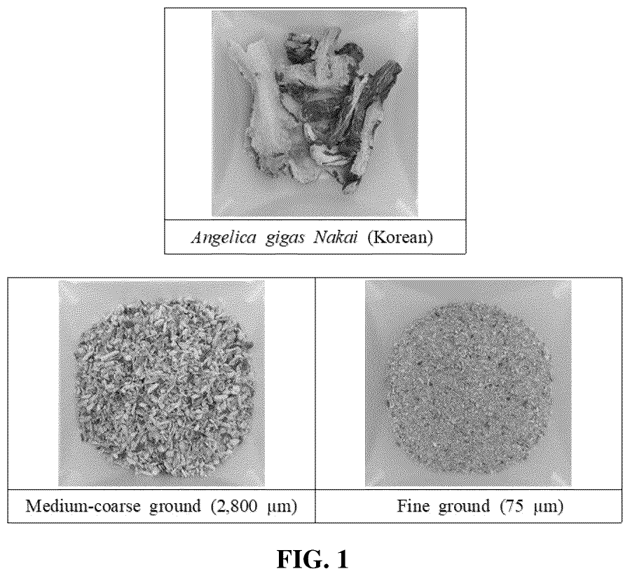

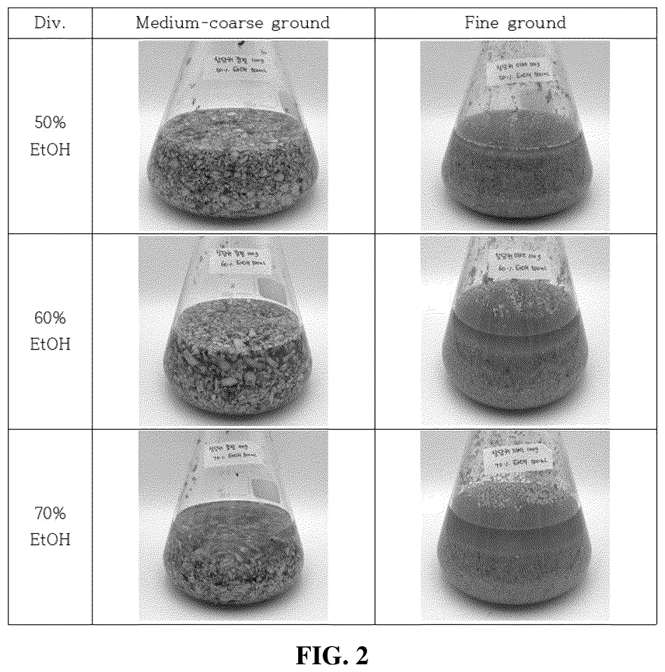

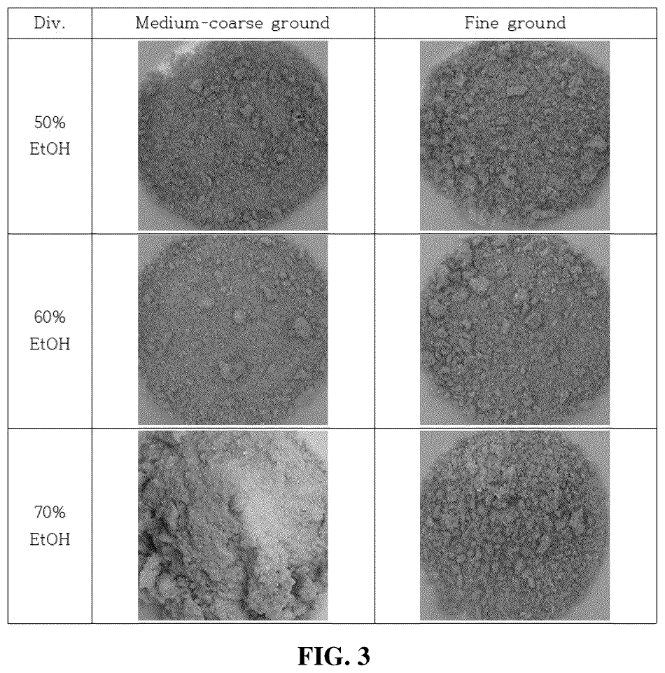

### DETAILED DESCRIPTION OF THE PREFERRED EMBODIMENT

Hereinafter, the present invention will be described in detail.

The terms used in the present invention are selected from commonly used general terms considering the functions of the present invention; however, they may vary depending on the intentions of those skilled in the art or the emergence of new technology in the field. In addition, there may be arbitrarily selected terms in specific cases, and in such cases, their meanings will be described in detail in the description of corresponding embodiments. Therefore, the terms used in the present invention should be defined based not only on the mere names of the terms but also on the meanings they carry and the overall content of the present invention.

In the present invention, when any component or step is “comprising” or “comprise”, it means that, unless specifically mentioned otherwise, it does not exclude other components or steps but rather allows for the inclusion of additional components or steps.

The present invention provides a method for preventing, improving or treating an inflammatory skin disease, the method comprising administering a composition comprising a fermented Angelica gigas extract as an active ingredient to a subject in need thereof.

In the present invention, the Angelica gigas may be Korean Angelica gigas Nakai but is not limited thereto.

In the present invention, the part of Angelica gigas for the fermented extract may be selected from the group consisting of leaves, stems, roots, and their mixtures of Angelica gigas. Preferably, it may be extracted from the roots of Angelica gigas but is not limited thereto.

In the present invention, the fermented extract may have an enhanced content of a main component selected from the group consisting of decursin, decursinol angelate, decursinol, and nodakenin.

In the present invention, the fermented extract may be prepared by a manufacturing method comprising the steps of: (a) grinding Angelica gigas; (b) fermenting the ground Angelica gigas by inoculating with a fermentation strain; and (c) extracting the extract by adding a solvent after drying the fermented product obtained in the above step (b).

The step (a) of grinding may be grinding to a size ranging from 10 m to 3,000 m, preferably in sizes of 20 m, 30 m, 40 m, 50 m, 60 m, 70 m, or greater; and it may also be grinding to a size less than 3,000 m, 2,900 m or 2,800 m, but it is not limited to these sizes.

In the present invention, the fermentation strain of the step (b) may be selected from the group consisting of Bacillus subtilis, Lactobacillus spp., Leuconostoc spp., Acetobacter sp., Leuconostoc spp., and Saccharomyces cerevisiae, but is not limited thereto.

In the present invention, the fermentation of the step (b) may be performed through solid-state fermentation or liquid-state fermentation.

The solid-state fermentation may involve preparing sterilized Angelica gigas with a moisture content of 40-60%, inoculating with fermentation strains and fermenting at a temperature of 22° C. to 35° C., preferably at 25° C. to 32° C., and more preferably at 30° C. Alternatively, fermentation may be performed at 30° C. for 16 hours and then at 25° C. for 8 hours. The fermentation time may range from 7 to 11 days, preferably 8 to 10 days, and more preferably around 9 days. Fermenting for less than 7 days may result in insufficient growth of the fermentation strains and hinder proper fermentation. Conversely, fermenting for more than 11 days may lead to the extinction of the fermentation strains, causing fermentation to occur ineffectively.

The liquid-state fermentation may involve adding Angelica gigas to an optimal growth medium for fermentation strains, sterilizing it, and inoculating with the fermentation strains. The fermentation may be then carried out at a temperature between 22° C. and 35° C., preferably between 25° C. and 32° C., and more preferably at 30° C. Alternatively, fermentation may be performed at 30° C. for 16 hours and then 25° C. for 8 hours. The fermentation time may vary from 12 hours to 3 days, preferably 1 to 3 days, and more preferably 1 to 2 days. It is worth noting that when the fermentation time is less than 12 hours, the growth of fermentation bacteria is insufficient, and fermentation does not occur effectively. Otherwise, when the fermentation time exceeds 3 days, the fermentation bacteria do not grow and disappear, leading to ineffective fermentation.

In the present invention, the solvent in step (c) may be selected from the group consisting of water, organic solvents, and mixtures thereof. The organic solvents may be selected from the group consisting of methanol, ethanol, propanol, isopropanol, butanol, acetone, ether, benzene, chloroform, ethyl acetate, methylene chloride, hexane, and cyclohexane. Preferably, the organic solvent may be 50-70% ethanol, but not limited thereto.

In the present invention, the extraction in step (c) may be carried out at room temperature, and the extraction time may be 36 to 60 hours, preferably 40 to 56 hours, 44 to 52 hours, 46 to 50 hours, or 47 to 49 hours, and more preferably 48 hours, but not limited thereto.

In the present invention, the inflammatory skin diseases may be selected from the group consisting of atopic dermatitis, contact dermatitis, allergic dermatitis, seborrheic dermatitis, erythematous lupus, urticaria, acne, eczema, and psoriasis, but are not limited thereto.

In the present invention, the amount of the active ingredient may be 0.0001 to 50 wt. %, preferably 0.001 to 30 wt. %, 0.001 to 20 wt. %, 0.01 to 20 wt. %, or 0.1 to 20 wt. % with respect to the total weight of the cosmetic composition, but is not limited thereto.

In the present invention, the composition is a cosmetic composition, a food composition or a pharmaceutical composition.

### Cosmetic Composition

The cosmetic composition of the present invention may include commonly used ingredients, including general adjuvants, e.g., antioxidants, stabilizers, solubilizers, vitamins, pigments, or fragrances, and carriers. It may further include functional additives, which may include a component selected from the group consisting of water-soluble vitamins, oil-soluble vitamins, polymer peptides, polymer polysaccharides, sphingolipids, and seaweed extracts. Furthermore, it may also include additive components, such as fat ingredients, moisturizers, emollients, surfactants, organic/inorganic pigments, organic powders, sunscreens, preservatives, disinfectants, antioxidants, plant extracts, pH adjusters, alcohols, dyes, fragrances, blood circulation enhancers, cooling agents, antiperspirants, or purified water.

The cosmetic composition of the present invention may be produced in any form commonly available in the related art. For example, it may be formulated as a solution, suspension, emulsion, paste, gel, cream, lotion, powder, soap, surfactant-containing cleanser, oil, powder foundation, emulsion foundation, wax foundation, or spray, but is not limited thereto. More specifically, the formulation may include a tonic, serum, skin lotion, skin softener, skin toner, astringent, lotion, milk lotion, moisturizing lotion, nourishing lotion, massage cream, nourishing cream, moisturizing cream, eye cream, hand cream, foundation, essence, nourishing essence, facial mask, soap, cleansing foam, cleansing lotion, cleansing cream, cleansing water, spray, powder, body lotion, or body wash.

When the formulation of the present invention is a paste, cream, or gel, the carrier component available herein may include: animal oils, vegetable oils, waxes, paraffin, starch, tragacanth, cellulose derivatives, polyethylene glycol, silicone, bentonite, silica, talc, or zinc oxide.

When the formulation of the present invention is a powder or spray, the carrier component available herein may include: lactose, talc, silica, aluminum hydroxide, calcium silicate, or polyamide powder. Particularly, in the case of spray formulations, a propellant such as chlorofluorocarbon, propane/butane, or dimethyl ether may be further included.

When the formulation of the present invention is a solution or suspension, the carrier component available herein may be a solvent, a solubilizer, or a suspending agent. Examples of the carrier component may include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid ester of sorbitan.

When the formulation of the present invention is a suspension, the carrier component available herein may include a liquid diluent, such as water, ethanol, or propylene glycol; a suspension, such as ethoxylated isostearyl alcohol, polyoxyethylene sorbitol ester; microcrystalline cellulose; aluminum metahydroxide; bentonite; agar; or tragacanth.

When the formulation of the present invention is a surfactant-containing cleanser, the carrier component available herein may include aliphatic alcohol sulfate, aliphatic alcohol ether sulfate, sulfosuccinate monoester, isethionate, imidazolinium derivative, methyltaurate, sarcosinate, fatty acid amide ether sulfate, alkylamido betaine, aliphatic alcohol, fatty acid glyceride, fatty acid diethanolamide, vegetable oil, lanolin derivative, or ethoxylated glycerol fatty acid ester.

### Food Composition

The food composition using a fermented Angelica gigas extract according to the present invention includes any form of a functional food, a nutritional supplement, a health food, or a food additive.

These types of food composition can be manufactured in various forms using methods commonly known in the related art.

For example, the food composition of the present invention as a health food may be manufactured in the form of tea, juice, and drink for consumption as it is. It may also be processed into granules, capsules, or powder for ingestion. Furthermore, the food composition of the present invention may be formulated into a composition in combination with a substance or active ingredient known for promoting hair growth and having anti-inflammatory effects.

As a functional food, the food composition of the present invention may be added to beverages (including alcoholic beverages), fruits and their processed foods (e.g., canned fruits, bottled fruits, jams, and marmalades), fish, meats and their processed foods (e.g., ham, sausages, and corned beef), breads, noodles (e.g., udon, buckwheat noodles, ramen, spaghetti, and macaroni), fruit juice, various drinks, cookies, taffy, dairy products (e.g., butter and cheese), edible plant oils, margarine, plant-based proteins, retort foods, frozen foods, or various seasonings (e.g., soybean paste, soy sauce, and sauces).

The content of the active ingredient of the present invention may be 0.01 to 50 wt. %, preferably 0.1 to 30 wt. %, 0.1 to 20 wt. %, or 1 to 20 wt. % with respect to the total weight of the food composition, but is not limited thereto.

For use in the form of a food additive, the food composition of the present invention may be manufactured in the form of a powder or concentrate.

### Pharmaceutical Composition

The pharmaceutical composition of the present invention may be formulated using a fermented Angelica gigas extract alone or in combination with a pharmaceutically acceptable carrier in an appropriate form. The composition may further contain excipients or diluents. The term ‘pharmaceutically acceptable’ as used herein refers to a non-toxic composition that is physiologically acceptable and does not cause allergic or similar reactions, such as gastrointestinal disorders or dizziness, when administered to humans.

The pharmaceutically acceptable carrier may include, for example, a carrier for oral or non-oral administration. The carrier for oral administration may include lactose, starch, cellulose derivatives, magnesium stearate, stearic acid, and the like. It may also include various drug delivery materials used for oral administration of peptide preparations. The carrier for non-oral administration may include water, suitable oils, saline solution, hydrophilic glucose, glycol, and the like, and may further include stabilizers and preservatives. The suitable stabilizers may include antioxidant agents, such as sodium hydrosulfite, sodium bisulfite, or ascorbic acid. The suitable preservatives may include benzalkonium chloride, methyl paraben, propyl paraben, or chlorobutanol. In addition to the above-mentioned components, the pharmaceutical composition of the present invention may further include lubricants, wetting agents, sweeteners, flavoring agents, emulsifiers, and glazing agents. Other pharmaceutically acceptable carriers and preparations may be referenced from the following literature: Remington's Pharmaceutical Sciences, 19th ed., Mack Publishing Company, Easton, PA, 1995.

The composition of the present invention may be administered to mammals, including humans, by any method. For example, it may be administered orally or non-orally. Non-oral administration methods are not limited to, but may include intravenous, intramuscular, intra-arterial, intramedullary, intrathecal, intracardiac, transdermal, subcutaneous, intraperitoneal, intranasal, intra-intestinal, local, rectal, or intra-rectal administration.

The pharmaceutical composition of the present invention may be formulated as an oral or non-oral preparation for administration according to the aforementioned routes.

For oral administration, the composition of the present invention may be formulated into powders, granules, tablets, suppositories, sugar-coated tablets, capsules, liquids, gels, syrups, slurries, suspensions, and other dosage forms, using methods known in the related art. For example, the oral preparation may be prepared by mixing active ingredients with a solid excipient, grinding the mixture, adding suitable excipients, and processing into a granule composite to obtain tablets or sugar-coated tablets. Examples of the suitable excipients may include sugars, e.g., lactose, dextrose, sucrose, sorbitol, mannitol, xylitol, erythritol, and maltitol; starches, e.g., corn starch, wheat starch, rice starch, and potato starch; celluloses, e.g., cellulose, methyl cellulose, sodium carboxymethyl cellulose, and hydroxypropyl methylcellulose; and fillers, e.g., gelatin and polyvinylpyrrolidone.

In some cases, cross-linked polyvinylpyrrolidone, carrageenan, alginic acid, or sodium alginate may be added as a disintegrant. Furthermore, the pharmaceutical composition of the present invention may also include anti-adhesive agents, lubricants, wetting agents, fragrances, emulsifiers, and preservatives.

For non-oral administration, the preparation may be formulated as injections, creams, lotions, topical ointments, oils, moisturizers, gels, aerosols, and nasal inhalants, according to the methods known in the related art. These formulations are described in the general prescription documents commonly known in pharmaceutical chemistry, such as Remington's Pharmaceutical Science, 19th edition, published by Mack Publishing Company, Easton, PA, 1995.

The total effective dose of the composition of the present invention may be administered to a patient as a single dose, or it may be administered over an extended period through a fractionated treatment protocol involving multiple doses. The pharmaceutical composition of the present invention may have the content of the active ingredients varied depending on the severity of the condition. The total daily dosage of the pharmaceutical composition of the present invention may be preferably 0.01 μg to 10,000 mg, and more preferably 0.1 μg to 500 mg, per 1 kg of the patient's body weight. However, the dosage of the pharmaceutical composition depends on various factors concerning the patient, such as age, weight, health status, gender, severity of the condition, diet, and excretion rate, as well as the method of preparation, route of administration, and frequency of treatment. Therefore, any one with ordinary skill in the art would be able to determine the appropriate effective dosage of the composition of the present invention, taking these factors into consideration. The pharmaceutical composition of the present invention is not limited to any specific formulation, route of administration, or method of administration as long as it exhibits the effects of the present invention.

Furthermore, the present invention provides an antimicrobial composition containing a fermented Angelica gigas extract as an active ingredient.

In the present invention, the antimicrobial composition may be effective against Gram-positive bacteria, Gram-negative bacteria or antibiotic-resistant bacteria, preferably any one selected from the group consisting of Staphylococcus, Listeria, Corynebacterium, Salmonella, Escherichia, Pseudomonas, Leptospira, and Rickettsia, and more preferably Staphylococcus aureus, but is not limited thereto.

Furthermore, the present invention provides an anti-inflammatory composition containing a fermented Angelica gigas extract as an active ingredient.

In the present invention, the fermented extract may inhibit the secretion or expression of inflammatory response factors selected from the group consisting of NO (Nitric oxide), iNOS (inducible Nitric oxide synthase), COX-2 (cyclooxygenase-2), and TNF-α (tumor necrosis factor-α).

Furthermore, the present invention provides a method for preparing a fermented Angelica gigas extract with an enhanced content of main components, comprising the steps of: (a) grinding Angelica gigas; (b) fermenting the ground Angelica gigas by inoculating with a fermentation strain; and (c) extracting the extract by adding a solvent after drying the fermented product obtained in the above step (b).

Hereinafter, the present invention will be described in more detail through examples. These examples are provided to explain the present invention more concretely, and the scope of the present invention is not limited to these examples.

### Example 1. Preparation of Fermented Angelica Gigas Extract

### 1.1. Preparation of Ethanolic Extract

In this study, Angelica Gigas roots were purchased from a domestic Korean herbal medicine store, Jecheon Herbal Medicine. The purchased herbal material was stored at room temperature and used for the experiments. For extraction, the herbal material was ground into coarse (2,800 m) or fine (75 m) particles, and 500 g of the ground material in each size was mixed with 50% ethanol, 60% ethanol, or 70% ethanol in 5-fold volume at room temperature for 48 hours (Method 1 and Method 2). The extracts were filtered and concentrated at 45° C. using a Rotary Vacuum Evaporator (Rotavapor R-215, BUCHI, Germany) (Method 3). The concentrated powder thus obtained was stored at −70° C. and used for the experiments. The yield of the concentrated powder was calculated according to the following formula:

Yield (%)=(Weight of the input material (g)/Weight of the dried powder (g))×100.

### 1.2. Fermentation Strains

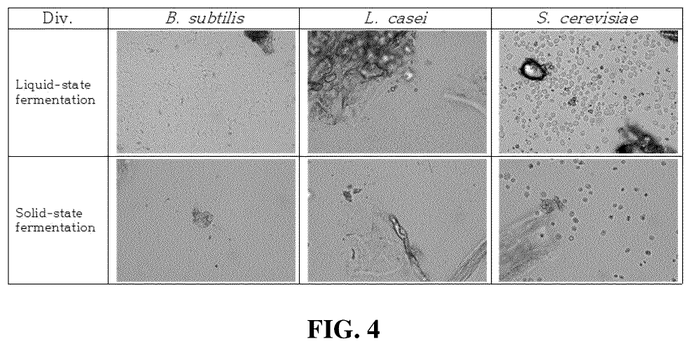

The fermentation strains of the present invention were obtained from Suwon University and used in the experiments as shown in Table 1, which included Bacillus subtilis, Lactobacillus casei, and Saccharomyces cerevisiae. Bacillus subtilis was incubated on nutrient agar (Difco Co., USA), Lactobacillus casei on MRS agar (Difco Co., USA), and Saccharomyces cerevisiae on yeast malt agar (Difco Co., USA). The fermentation methods and microscopic images of each fermented strain are presented in FIG. 4.

TABLE 1

| Strains | Sources |
|---|---|
| Bacillus subtilis | Doenjang |
| Lactobacillus casei | Cheese |
| Saccharomyces cerevisiae | Grapes |

### 1.3. Preparation of Fermented Angelica Gigas Extract

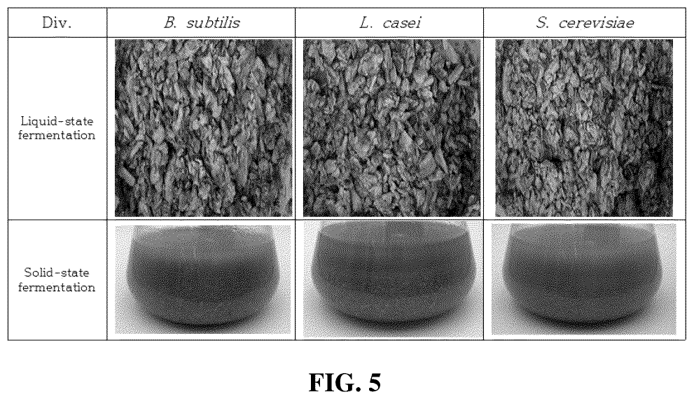

For liquid-state fermentation, 7.5 g of Angelica gigas was added to 150 mL of optimal growth medium for each strain and sterilized. Then, it was inoculated with 1% (v/v) of the culture and fermented at a temperature of 30° C. or under alternating temperature conditions (30° C. for 8 hours, 25° C. for 16 hours) for 1 or 2 days (FIG. 5). Subsequently, the fermented Angelica gigas was dried in a dryer at 50° C. for 2 days and extracted with 50 mL of 70% ethanol at room temperature for 48 hours. The non-fermented Angelica gigas was extracted with 50 mL of 70% ethanol at room temperature for 48 hours, and the resultant extract was used as a control group.

For solid-state fermentation, 50 g of Angelica gigas was sterilized with an initial moisture content of 50% and cooled down to the room temperature. It was then inoculated uniformly with 50 mL of each fermentation strain and fermented at a temperature of 30° C. or under alternating temperature conditions (30° C. for 8 hours, 25° C. for 16 hours) (FIG. 5). After fermentation, the fermented Angelica gigas was extracted at the room temperature for 48 hours with 70% ethanol added in a volume that was 5 times higher than the volume of the fermented Angelica gigas. The non-fermented Angelica gigas was extracted at the room temperature for 48 hours with 70% ethanol added in a volume that was 10 times higher than the volume of non-fermented Angelica gigas, and the resultant extract was used as a control group.

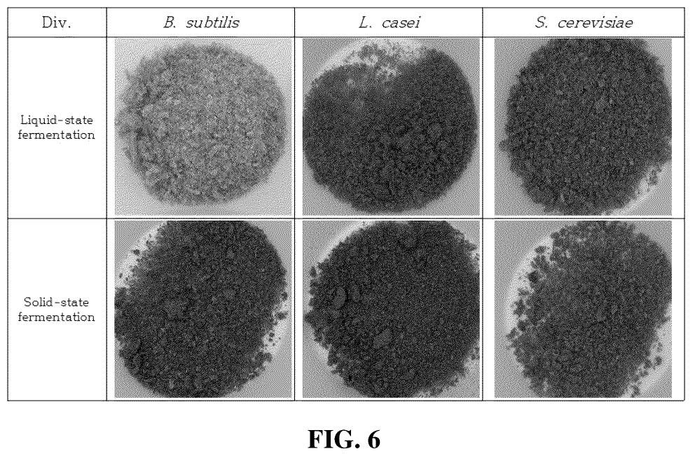

Both the liquid-state fermented extract and the solid-state fermented extract were filtered and concentrated with a rotary vacuum evaporator (Rotavapor R-215, BUCHI, Germany) at 45° C. (FIG. 6). The concentrated powder was stored at −70° C. and used for the experiments.

### Example 2. Analysis on Active Ingredient Content of Fermented Angelica Gigas Extract

### 2.1. HPLC (High-Performance Liquid Chromatography) Analysis Method

The content of the active ingredients, decursin, decursinol angelate, decursinol, and nodakenin in the fermented Angelica gigas extract was determined through HPLC analysis. The concentrated powder sample of each extract was dissolved in methanol to a concentration of 1,000 ppm and filtered through a 0.45 m syringe filter before use. For standard forms, decursin, decursinol, decursinol angelate, and nodakenin (Sigma, USA) were dissolved and diluted to various concentrations to obtain standard curves for use in the experiment. The analytical instrument was an HPLC Agilent LC-P-10 pump with a Shiseido C18 column (UG120, 5 m, 4.6 I.D.×250 mm). 5 μL of each sample was injected and separated by isocratic elution with 50% acetonitrile (10 mM sodium dodecyl sulfate, 25 mM sodium phosphate dibasic) at a flow rate of 0.7 mL/min. Detection was monitored at 330 nm using ultraviolet (UV) light. The HPLC analysis conditions for the fermented Angelica gigas extract are shown in Table 2 below.

TABLE 2

| Instrument | Conditions | |
|---|---|---|
| Column | Shiseido C18 (4.6 × 250 mm, 5 μm) | |
| Mobile phase | 50% Acetonitrile | |
| | (10 mM Sodium Dodecyl Sulfate, | |
| | 25 mM Sodium phosphate dibasic) | |
| Detector | 330 | nm |
| Flow rate | 0.7 | mL/min |
| Column temperature | 30° | C. |
| Injection volume | 5 | μL |
| Runtime | 30 | min |

### 2.2. Results

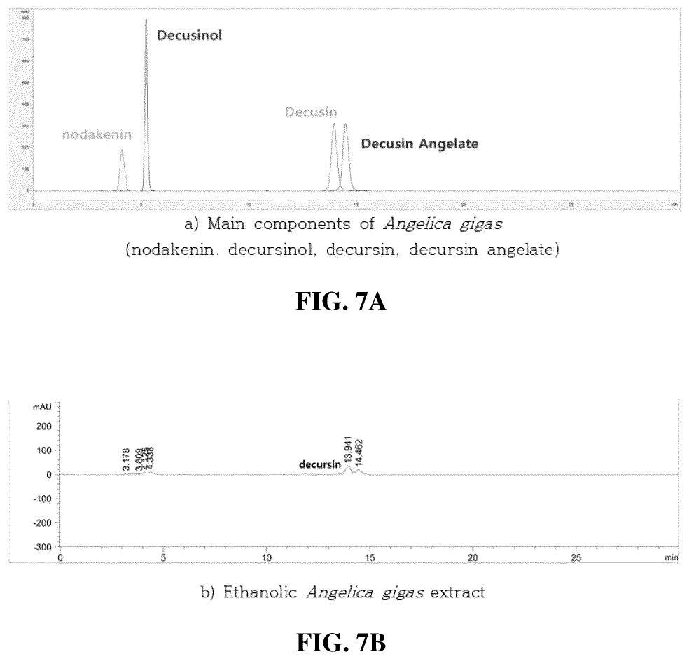

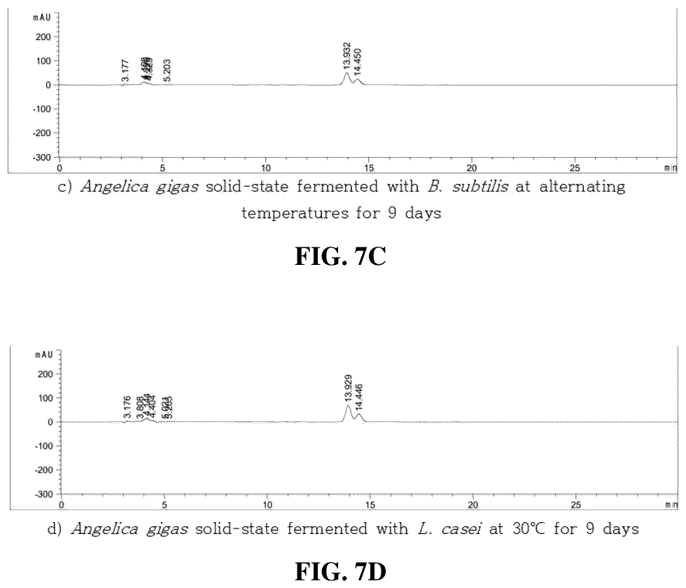

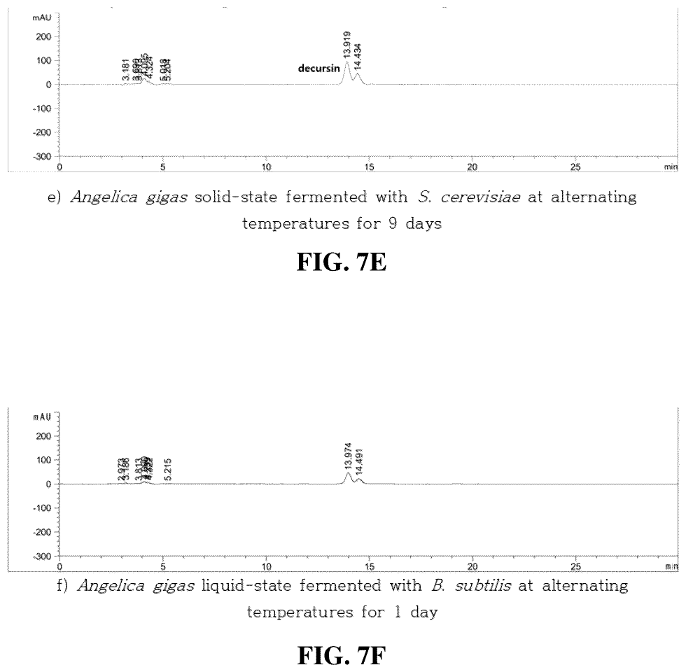

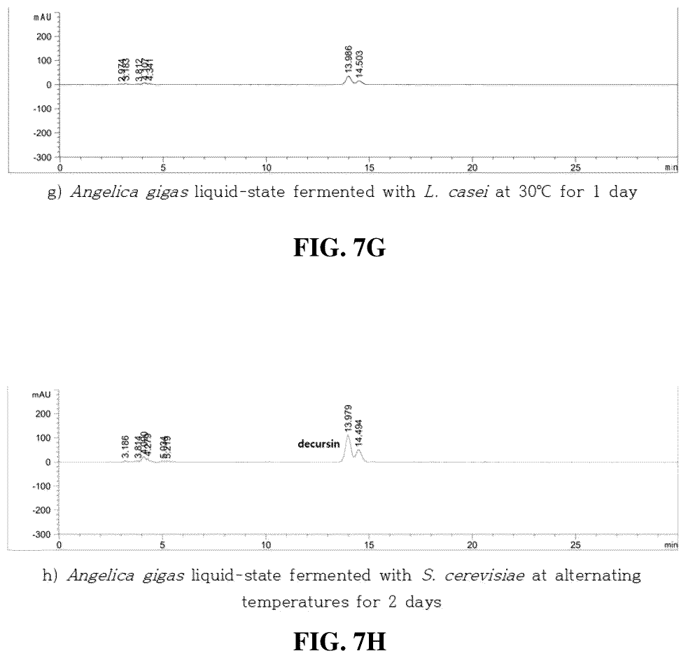

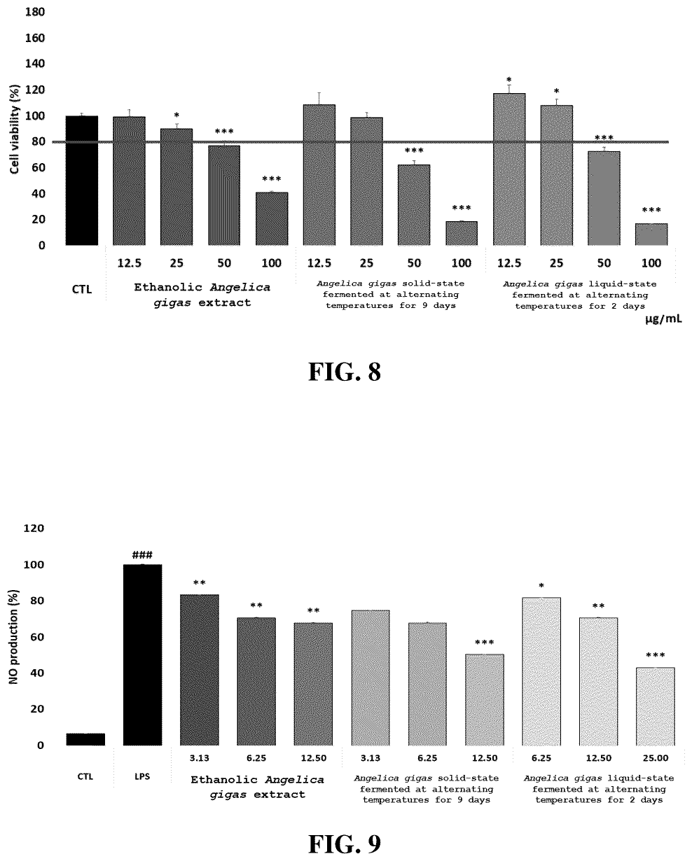

The contents of active components, decursin, decursinol angelate, decursinol, and nodakenin, of the extracts before and after fermentation of Angelica gigas were determined as shown in FIGS. 7A-7H and Table 3, where the contents of active ingredients were all higher in the fermented Angelica gigas extract than in the non-fermented control group.

Specifically, the non-fermented control group contained 47.16 mg/g of decursin, 25.25 mg/g of decursinol angelate, 0.88 mg/g of decursinol, and 14.519 mg/g of nodakenin. In contrast, the fermented extract obtained by solid-state fermentation using B. subtilis under alternating temperature conditions (30° C. for 8 hours and 25° C. for 16 hours) for 9 days had 69.48 mg/g of decursin, 31.95 mg/g of decursinol angelate, 2.11 mg/g of decursinol, and 15.47 mg/g of nodakenin. The fermented extract obtained by liquid-state fermentation under alternating temperature conditions for 1 day contained 94.81 mg/g of decursin, 28.73 mg/g of decursinol angelate, 2.13 mg/g of decursinol, and 15.59 mg/g of nodakenin.

Further, the fermented extract obtained by solid-state fermentation using L. casei at 30° C. for 9 days contained 96.04 mg/g of decursin, 44.59 mg/g of decursinol angelate, 2.368 mg/g of decursinol, and 30.30 mg/g of nodakenin. The fermented extract obtained by liquid-state fermentation at 30° C. for 1 day contained 48.20 mg/g of decursin, 20.35 mg/g of decursinol angelate, 1.17 mg/g of decursinol, and 15.35 mg/g of nodakenin.

Furthermore, the fermented extract obtained by solid-state fermentation using S. cerevisiae under alternating temperature conditions for 9 days contained 130.68 mg/g of decursin, 61.179 mg/g of decursinol angelate, 3.82 mg/g of decursinol, and 44.07 mg/g of nodakenin. The fermented extract obtained by liquid-state fermentation using S. cerevisiae under alternating temperature conditions for 2 days contained 152.17 mg/g of decursin, 19.07 mg/g of decursinol angelate, 4.68 mg/g of decursinol, and 35.92 mg/g of nodakenin.

It was confirmed from the above results that the contents of the active ingredients such as decursin, decursinol angelate, decursinol, and nodakenin were all higher in the fermented Angelica gigas extracts than in the non-fermented control group. In particular, the solid-state and liquid-state fermented extracts had the decursin content around 2.8 times and 3.2 times far higher than the control group, respectively.

TABLE 3

| | | | | | Compound (mg/g) | | | |
|---|---|---|---|---|---|---|---|---|
| | Fermentation | | Temp. | | | Decursinol | | |
| Plant | Strains | Methods | (° C.) | Days | Decursin | angelate | Decursinol | Nodakenin |
| Angelica | — | — | — | — | 47.16 | 25.25 | 0.88 | 14.519 |
| gigas | B. subtilis | Solid | Alternating | 9 | 69.48 | 31.95 | 2.11 | 15.47 |
| | | Liquid | Alternating | 1 | 94.81 | 28.73 | 2.13 | 15.59 |
| | L. casei | Solid | 30 | 9 | 96.04 | 44.59 | 2.368 | 30.30 |
| | | Liquid | 30 | 1 | 48.20 | 20.35 | 1.17 | 15.35 |
| | S. cerevisiae | Solid | Alternating | 9 | 130.68 | 61.179 | 3.82 | 44.07 |
| | | Liquid | Alternating | 2 | 152.17 | 19.07 | 4.68 | 35.92 |

### Example 3. Antimicrobial Activity of Fermented Angelica Gigas Extract

### 3.1. Measurement Method of Antimicrobial Activity

The strain used for measuring the antimicrobial activity was Staphylococcus aureus subsp. aureus KCTC 3881, supplied from KCTC (Korean Collection for Type Cultures) and incubated on tryptic soy agar (Difco Co., USA) at 37° C.

The antimicrobial activity of each extract was determined according to the agar diffusion method using paper discs with a growth medium incubated by the mixed plate method. Each extract was prepared to concentrations of 2% and 4% using sterilized water and filtered through a syringe filter (0.4 m, ADVANTEC, Toyo Roshi Kaisha, Ltd., Japan) for experimental use. The strain was incubated on an optimal growth medium using the mixed plate method, and 20 μL of the extract was placed over paper discs (8 mm, ADVANTEC, Toyo Roshi Kaisha, Ltd., Japan). After 24 hours of incubation at the optimal growth temperature, the presence/absence and size of a ‘clear zone’ was measured to evaluate the antimicrobial activity.

### 3.2. Results

The antimicrobial activity of Angelica gigas against Staphylococcus aureus was analyzed under different conditions, including the extraction method, fermentation method, fermentation temperature, and fermentation duration. The results, as shown in Table 4 below, indicated that the non-fermented control group had a clear zone with a diameter of 8.5±0 mm at 2% concentration and 9±0.29 mm at 4% concentration. In contrast, the fermented extract obtained by solid-state fermentation using B. subtilis under alternating temperature conditions for 9 days had a clear zone with a diameter of 8.5±0 mm at 2% concentration and 9.17±0.33 mm at 4% concentration. Further, the fermented extract obtained by liquid-state fermentation under alternating temperature conditions for 1 day had a clear zone with a diameter of 8.5±0 mm at 2% concentration and 9.25±0.25 mm at 4% concentration.

Furthermore, the fermented extract obtained by solid-state fermentation using L. casei at 30° C. for 9 days had a clear zone with a diameter of 8.83±0.17 mm at 2% concentration and 9.69±0.17 mm at 4% concentration. The fermented extract obtained by liquid-state fermentation at 30° C. for 1 day had a clear zone with a diameter of 8.5±0 mm at 2% concentration and 9.33±0.17 mm at 4% concentration. Furthermore, the fermented extract obtained by solid-state fermentation using S. cerevisiae under alternating temperature conditions for 9 days had a clear zone with a diameter of 9.67±0.17 mm at 2% concentration and 10±0 mm at 4% concentration. The fermented extract obtained by liquid-state fermentation under alternating temperature conditions for 2 days had a clear zone with a diameter of 9.5±0 mm at 2% concentration and 9.5±0 mm at 4% concentration.

It was confirmed from the above results that the fermented Angelica gigas extracts exhibited significantly high antimicrobial activity at 4% concentration, particularly the fermented extract obtained by solid-state or liquid-state fermentation using S. cerevisiae showed significantly high antimicrobial activity at both 2% and 4% concentrations.

Accordingly, the fermented Angelica gigas extract of the present invention proved to be usable as an antimicrobial composition through its antimicrobial activity, especially against Staphylococcus aureus.

TABLE 4

| | Fermentation | | Temperature | | 2% | 4% |
|---|---|---|---|---|---|---|
| Plant | Strain | Method | (° C.) | Days | Clear zone (mm, Ø) | |
| Angelica | — | — | — | — | 8.5 ± 0 | 9 ± 0.29 |
| gigas | B. subtilis | Solid | Alternating | 9 | 8.5 ± 0 | 9.17 ± 0.33 |
| | | Liquid | Alternating | 1 | 8.5 ± 0 | 9.25 ± 0.25 |
| | L. casei | Solid | 30 | 9 | 8.83 ± 0.17 | 9.69 ± 0.17 |
| | | Liquid | 30 | 1 | 8.5 ± 0 | 9.33 ± 0.17 |
| | S. cerevisiae | Solid | Alternating | 9 | 9.67 ± 0.17 | 10 ± 0 |
| | | Liquid | Alternating | 2 | 9.5 ± 0 | 9.5 ± 0 |

### Example 4. Evaluation of Cytotocisity

### 4.1. MTT Assay

The cytotoxicity of the fermented Angelica gigas extract was determined using the MTT assay. To perform the MTT assay, HaCaT cells, previously incubated for 24 hours, were treated with lipopolysaccharide (LPS) and extract samples at different concentrations (12.5, 25, 50, and 100 ppm) simultaneously and incubated for another 24 hours. Subsequently, 500 μg/mL of MTT (3-(4,5-dimethylthiazol-2-yl)-2,5-diphenyltetrazolium bromide) was added to the cells, which were then incubated at 37° C. in a 5% CO₂ incubator for 4 hours. The MTT solution was then removed, and DMSO was added to dissolve the formed formazan precipitate, which resulted from the reaction of viable cells. The absorbance was measured at 540 nm. The cell viability (%) was calculated as given by the following formula:

Cell viability (%)=100×{Absorbance of control/Absorbance of tested extract solution}.

### 4.2. Results

The groups treated with the ethanolic extracts using non-fermented Angelica gigas at concentration of 12.5, 25, 50, and 100 ppm showed cell viability of 99.52±5.17%, 90.10±3.95%, 77.34±3.67%, and 40.88±1.22%, respectively, with respect to the untreated control group (100%). Treating with the fermented extract obtained by solid-state fermentation under alternating temperature conditions for 9 days (Angelica gigas solid-state fermented at alternating temperatures for 9 days) resulted in cell viability of 108.46±9.29%, 98.94±3.87%, 62.60±3.15%, and 18.89±0.70% at 12.5, 25, 50, and 100 ppm, respectively. In addition, when treating with the fermented extract obtained by liquid-state fermentation under alternating temperature conditions for 2 days (Angelica gigas liquid-state fermented at alternating temperatures for 2 days), the cell viability was 117.31±6.78%, 108.43±4.44%, 72.83±3.08%, and 17.26±0.11% at 12.5, 25, 50, and 100 ppm, respectively.

Based on the results, indicating that there was no cytoxicity at concentrations with a cell survival rate of over 100%, the ethanolic Angelica gigas extract and the fermented Angelica gigas extract obtained by solid-state fermentation under alternating temperature conditions for 9 days exhibited no cytoxicity at concentrations of up to 12.5 ppm. Similarly, the fermented Angelica gigas extract obtained by liquid-state fermentation under alternating temperature conditions for 2 days showed no cytoxicity at concentrations of up to 12.5 ppm. Therefore, the maximum concentration at which no cell toxicity was observed was used as the reference for conducting the subsequent experiments.

### Example 5. Anti-Inflammatory Activity of Fermented Angelica Gigas Extract

### 5.1. Measurement Method of NO (Nitric Oxide) Production

RAW 264.7 cells were seeded at a density of 1×105 cells/well and incubated at 37° C. under 5% CO₂ conditions for 18 hours. After removing the medium, the cells were treated with a medium containing 100 ng/mL of LPS (Sigma, USA) and incubated for additional 24 hours with various sample concentrations. Subsequently, 100 μL of cell culture supernatant and 100 μL of Griess reagent (1% sulfanilamide, 0.1% naphthylethylenediamine in 2.5% phosphoric acid) were mixed and allowed to react for 10 minutes in a 96-well plate. The absorbance was measured at 540 nm to determine the amount of generated NO. The NO content in the cell culture supernatant was measured in the form of NO₂⁻ using the Griess reagent, and sodium nitrite (NaNO₂) was used as the standard for quantification.

### 5.2. Results

To investigate the inhibitory effects of each extract against the inflammation-mediating factors activated by LPS (lipopolysaccharide)-stimulated cells, the nitric oxide (NO) inhibitory activity of each extract was analyzed. Based on the cytotoxicity results, the concentrations of each extract were set as follows: 3.13 ppm, 6.25 ppm and 12.50 ppm for the fermented extract obtained by solid-state fermentation under alternating temperature conditions for 9 days (Angelica gigas solid-state fermented at alternating temperatures for 9 days); and 6.25 ppm, 12.50 ppm and 25.00 ppm for the fermented extract obtained by liquid-state fermentation under alternating temperature conditions for 2 days (Angelica gigas liquid-state fermented at alternating temperatures for 2 days).

As a result, the ethanolic extract of Angelica gigas showed an NO production of 83.44±0.13%, 70.78±0.22% and 67.76±0.22% at concentrations of 3.13 ppm, 6.25 ppm, and 12.50 ppm, respectively. The fermented extract obtained by solid-state fermentation under alternating temperature conditions for 9 days (Angelica gigas solid-state fermented at alternating temperatures for 9 days) exhibited an NO production of 74.68±0.13%, 67.86±0.38% and 50.32±0.22% of NO generation at concentrations of 3.13 ppm, 6.25 ppm and 12.50 ppm, respectively. Further, the fermented extract obtained by liquid-state fermentation under alternating temperature conditions for 2 days (Angelica gigas liquid-state fermented at alternating temperatures for 2 days) presented an NO production of 81.76±0.13%, 70.61±0.22% and 43.24±0.00% at concentrations of 6.25 ppm, 12.50 ppm and 25.00 ppm, respectively.

It was confirmed from the above results that the fermented Angelica gigas extract has concentration-dependent NO inhibitory activity, indicting potential effects for prevention or treatment of inflammatory diseases through its anti-inflammatory activity.

The description of the invention provided above is for illustrative purposes and should be understood by those skilled in the art that the technical concept or essential features of the invention can be easily modified into other specific forms without altering the scope of the invention. Therefore, the embodiments described above should be considered exemplary in all respects and not limiting. For example, each component described as a single form can also be implemented in a distributed manner, and similarly, components described as distributed can also be combined in a unified form.

The scope of the invention is defined by the claims set forth below, and all modifications or variations derived from the meaning and scope of the claims, as well as the concept of equivalence, should be interpreted as falling within the scope of the invention.

What is claimed is:

1. A method for preventing, improving or treating an inflammatory skin disease, the method comprising administering a composition comprising a fermented Angelica gigas extract as an active ingredient to a subject in need thereof.

2. The method of claim 1, wherein the fermented extract is enhanced in the content of a main component selected from the group consisting of decursin, decursinol angelate, decursinol, and nodakenin.

3. The method of claim 1, wherein the fermented extract is prepared by a method comprising:
(a) grinding Angelica gigas;
(b) fermenting the ground Angelica gigas by inoculating with a fermentation strain; and
(c) extracting the extract by adding a solvent after drying the fermented product obtained in the above step (b).

4. The method of claim 3, wherein the fermentation strain of the step (b) is selected from the group consisting of Bacillus subtilis, Lactobacillus spp., Leuconostoc spp., Acetobacter spp., Leuconostoc spp., and Saccharomyces cerevisiae.

5. The method of claim 3, wherein the fermentation of the step (b) is solid-state fermentation or liquid-state fermentation.

6. The method of claim 5, wherein the solid-state fermentation comprises preparing Angelica gigas with a moisture content of 40-60%, sterilizing, inoculating with a fermentation strain and fermenting at a temperature of 25° C. to 30° C. for 7 to 11 days.

7. The method of claim 5, wherein the liquid-state fermentation comprises adding Angelica gigas to an optimal growth medium for a fermentation strain, sterilizing, inoculating with the fermentation strain, and fermenting at 25° C. to 30° C. for 12 hours to 3 days.

8. The method of claim 3, wherein the solvent of the step (c) is selected from the group consisting of water, an organic solvent, and a mixture thereof.

9. The method of claim 8, wherein the organic solvent is selected from the group consisting of methanol, ethanol, propanol, isopropanol, butanol, acetone, ether, benzene, chloroform, ethyl acetate, methylene chloride, hexane, and cyclohexane.

10. The method of claim 3, wherein the extraction of the step (c) is performed at room temperature for 36 to 60 hours.

11. The method of claim 1, wherein the inflammatory skin disease is selected from the group consisting of atopic dermatitis, contact dermatitis, allergic dermatitis, seborrheic dermatitis, erythematous lupus, urticaria, acne, eczema, and psoriasis.

12. The method of claim 1, wherein the composition is a cosmetic composition, a food composition or a pharmaceutical composition.

13. An antimicrobial composition comprising a fermented Angelica gigas extract as an active ingredient.

14. An anti-inflammatory composition comprising a fermented Angelica gigas extract as an active ingredient.

15. A method for preparing a fermented Angelica gigas extract with an enhanced content of a main component, comprising:
(a) grinding Angelica gigas;
(b) fermenting the ground Angelica gigas by inoculating with a fermentation strain; and
(c) extracting the extract by adding a solvent after drying the fermented product obtained in the above step (b).
